(19) United States
(12) Patent Application Publication
(10) Pub. No.: US 2011/0300086 A1
(43) Pub. Date: Dec. 8, 2011

(54) Film-Forming Solutions Comprising Vitamin D or Derivative Thereof and a Corticosteroid and Dermatological Applications Thereof

(75) Inventors: Claire MALLARD, Mougins (FR); Laurent Fredon, Roquefort Les Pins (FR)

(73) Assignee: GALDERMA S.A., CHAM (CH)

(21) Appl. No.: 12/983,608

(22) Filed: Jan. 3, 2011

Related U.S. Application Data
(63) Continuation of application No. 11/976,066, filed on Oct. 19, 2007, now abandoned.
Application No. 11/976,066 is a continuation of application No. PCT/EP2006/004315, filed on Apr. 14, 2006.
(60) Provisional application No. 60/676,291, filed on May 2, 2005.

(30) Foreign Application Priority Data
Apr. 19, 2005 (FR) 0503913

(51) Int. Cl. A61K 31/59; A61K 31/592; A61P 17/06; A61K 31/593

(52) U.S. Cl. 424/61; 514/167

(57) ABSTRACT

Topically applicable film-forming solutions, e.g., nail varnishes, useful for such dermatological applications as the prevention or treatment of nail psoriasis, contain, as active agents solubilized therein, vitamin D or derivative thereof and a corticosteroid, formulated into a topically applicable, physiologically acceptable medium therefor.

### CROSS-REFERENCE TO PRIORITY/PCT/PROVISIONAL APPLICATIONS

This application is a continuation of application Ser. No. 11/976,066, filed Oct. 19, 2007, which in turn claims priority under 35 U.S.C. §119 of FR 0503913, filed Apr. 19, 2005, and of Provisional Application No. 60/676,291, filed May 2, 2005, and is a continuation of PCT/EP 2006/004315, filed Apr. 14, 2006 and designating the United States, published in the English language as WO 2006/111426 A1 on Oct. 26, 2006, each hereby expressly incorporated by reference in its entirety and each assigned to the assignee hereof.

### BACKGROUND OF THE INVENTION

1. Technical Field of the Invention

The present invention relates to novel compositions of film-forming solution type, useful for application to the nails, comprising two solubilized active agents which are:
vitamin D or a derivative thereof, preferably calcitriol, and
a corticosteroid, preferably clobetasol 17-propionate, and also to the use thereof in dermatology for the treatment of nail psoriasis.

2. Description of Background and/or Related and/or Prior Art

Psoriasis is a chronic inflammatory disease of the skin which affects approximately 5% of the French population. This disease manifests itself through lesions, promoting quite distinct forms of psoriasis.

Among the latter, nail psoriasis involves approximately half the individuals suffering from the disease. The nails of the hands are affected more than those of the feet. The nail, which grows in an accelerated manner, is then subjected to certain alterations—due to keratinization problems—depending on the location of the psoriasis. In certain instances, it exhibits at its surface small depressions which give it a thimble-like appearance. Sometimes, salmon-colored marks appear, or even a discoloration. The nail can also undergo thickening which can detach it from its bed. Finally, it sometimes exhibits transverse or longitudinal striations. The inflammation can also extend under the nail: in this case, the lesions are not in contact with the air and heal with difficulty.

This pathology in the nail is generally difficult to treat, even more so since few treatments are available. Among the current treatments proposed for topical administration, mention may, by way of example, be made of:
calcipotriol solution;
cyclosporin or anthralin ointment (“Treatment of psoriatic nails with topical cyclosporin: a prospective, randomized placebo-controlled study,” Cannavo et al., Dermatology 2003; 206 (2): 153-6);
the application of a class I corticosteroid;
5-fluorouracil cream.

Antifungal treatments can also be prescribed in the case of secondary infection.

None of these treatments is very effective. Thus, other routes and combinations have been researched, for example:
tazarotene gel (“Tazarotene 0.1% gel in the treatment of fingernail psoriasis: a double-blind, randomized, vehicle-controlled study,” Scher R. K. et al., Cutis 2001 November; 68 (5) 355-8 and “Tazarotene 0.1% gel for psoriasis of the fingernails and toenails: an open, prospective study,” Bianchi Let al., Br. J. Dermatol., 2003 July; 149 (1) : 207-9);
the combination of cyclosporin administered orally with a calcipotriol cream (“Nail psoriasis: combined therapy with systemic cyclosporin and topical calcipotriol,” Feliciani et al., J Cutaneous Medicine Surgery: Incorporating Medical and Surgical Dermatology, 2004; 8 (2): 122-5);
combination, by topical administration, of a calcitriol cream and a clobetasol 17-propionate cream (“Nail psoriasis: a combined treatment using calcipotriol cream and clobetasol propionate cream,” Rigopoulos et al., Acta Derm Venereol., 2002; 82(2): 140).

However, the combination of active ingredients is not administered conventionally in the treatment of dermatological conditions. The difficulties mainly encountered by those skilled in the art when combining two active ingredients are the problems of chemical instability and interactions that the active ingredients may exhibit when they are present in the same formulation.

Vitamin D and derivatives thereof are unstable in aqueous media, and sensitive to acidic pHs, whereas corticosteroids, and more particularly clobetasol 17-propionate, are themselves sensitive to basic media.

The assignee hereof has described, in FR-2,848,454, a combination of calcitriol with a corticosteroid in the treatment of certain dermatological conditions, without however proposing any stable pharmaceutical compositions combining the two active agents.

Moreover, in the field of dermatology and of the formulation of pharmaceutical compositions, those skilled in the art seek compositions which not only must be physically and chemically stable, but also must make it possible to release the active agent and to promote the penetration thereof over the target zone in order to improve its effectiveness.

### SUMMARY OF THE INVENTION

The present invention features specific compositions for the nails, comprising vitamin D or a derivative thereof and a corticosteroid, said compositions being stable and well tolerated.

Thus, this invention features novel formulations, of film-forming solution type, comprising vitamin D or a derivative thereof, preferably calcitriol, combined with a corticosteroid, preferably clobetasol 17-propionate. The subject compositions are useful in the treatment of nail psoriasis. The solution form allows local application of the treatment without systemic exposure; it is therefore well tolerated. Furthermore, by virtue of its composition, such solutions permit a gradual release of the active ingredients.

The present invention therefore features compositions comprising, formulated into a physiologically acceptable medium, as pharmaceutical active agents, vitamin D or a derivative thereof and a corticosteroid, wherein said compositions are film-forming solutions. These solutions are preferably nail varnishes.

### DETAILED DESCRIPTION OF BEST MODE AND SPECIFIC/PREFERRED EMBODIMENTS OF THE INVENTION

The present invention preferably relates to compositions comprising, formulated into a physiologically acceptable medium, as pharmaceutical active agents, calcitriol and clobetasol 17-propionate, said compositions being film-forming solutions, preferably nail varnishes.

In the text which follows, clobetasol 17-propionate will be referred to as clobetasol propionate.

The term “physiologically acceptable medium” means any medium that is compatible with the skin, the mucous membranes and the integuments.

The term “film-forming solution” means a solution containing at least one film-forming polymer. Such a solution is preferably suited for application to the nails.

Preferably, the film-forming solutions according to the invention are nail varnishes.

Advantageously, the film-forming solutions according to the invention are non-aqueous solutions. The term “non-aqueous solution” means a solution free of added water. The solution may, however, contain an amount of residual water not exceeding 5% of the total concentration of solvents/cosolvents of the composition. Preferably, the film-forming solution according to the invention is a non-aqueous nail varnish.

Such compositions are therefore for topical application.

Such compositions can also be sprayed with or without propellant gas.

When the composition contains a propellant gas, it is selected from the group consisting of propane, butane, isobutane, dichlorodifluoromethane, dichlorotetrafluoroethane, octafluorocyclobutane, nitrogen, CO₂ and dimethyl ether, or mixtures thereof.

According to a preferred embodiment of the invention, the propellant gas is in liquefied form and its concentration is from 5% to 30% of the total composition.

According to a preferred embodiment of the invention, the composition is a film-forming solution, preferably a nail varnish, which comprises, formulated into a physiologically acceptable medium, as pharmaceutical active agents, vitamin D or a derivative thereof and a corticosteroid, which are present in solubilized form.

The term “solubilized form” means a dispersion in the molecular state in a liquid, no crystallization of the active agents being visible to the naked eye nor even under a cross-polarized optical microscope.

In the subsequent text, the amounts are expressed as percentage by weight relative to the total weight of the composition (m/m).

In one embodiment of the invention, the film-forming solutions as defined above comprise:
a) vitamin D or a derivative thereof and a corticosteroid, which are solubilized,
b) an organic solvent/cosolvent mixture, and
c) at least one film-forming agent.

In a preferred embodiment of the invention, the film-forming solutions as defined above comprise:
a) calcitriol and clobetasol propionate, which are solubilized,
b) an organic solvent/cosolvent mixture, and
c) at least one film-forming agent.

The term “vitamin D” means the various forms of vitamin D, such as, for example, vitamin D₁, D₂, D₃ or vitamin D₄.

The term “vitamin D derivatives” means compounds which exhibit biological properties similar to those of vitamin D, in particular vitamin D response element (VDRE) transactivating properties, such as an agonist or antagonist activity with respect to vitamin D receptors. These compounds are not generally natural metabolites of vitamin D, but are in particular synthetic compounds comprising the vitamin D backbone with modifications on the side chains and/or comprising modifications in the backbone itself.

Among vitamin D derivatives, exemplary are calcipotriol, 25-hydroxyvitamin D₃, 1α-hydroxyvitamin D₃, calcitriol or 1α, 25-dihydroxyvitamin D₃, 1α, 25, 2β-trihydroxy-vitamin D3, 1α, 23, 25-trihydroxyvitamin D3, 24, 25-dihydroxyvitamin D3, 1α, 25-dihydroxyvitamin D2, 1α-hydroxyvitamin D2, 1α, 24-dihydroxyvitamin D2 and 1α,24-dihydroxyvitamin D3 (or tacalcitol), and mixtures thereof.

According to a preferred embodiment of the invention, the vitamin D derivative is calcitriol.

The amount of vitamin D or derivatives thereof that can be incorporated according to the invention is from 0.00001% to 0.1% m/m, preferably from 0.0001% to 0.001% m/m, and preferably from 0.0002% to 0.0005% m/m. This amount is preferably equal to 0.0003% m/m.

Among the corticosteroids, exemplary are clobetasone and esters thereof such as the 17-butyrate, clobetasol and esters thereof such as the 17-propionate, hydrocortisone and esters thereof such as the 17-butyrate, cortisone and esters thereof such as the 21-acetate, prednisolone and esters thereof such as pivalate, miconazole, prednisone, triamcinolone and esters and ethers thereof such as triamcinolone acetonide, methylprednisolone, fluometholone, fluocinolone and esters and ethers thereof such as fluocinolone acetonide, desonite, betamethasone and esters thereof such as the 21-acetate, the 17-adamantoate, the 17-benzoate, the 17-valerate and the 17, 21-di[rho]ropionate, and dexamethasone, and mixtures and derivatives thereof.

The term “corticosteroid derivatives” is intended in particular to mean their pharmaceutically acceptable salts with a base, such as the disodium phosphate salts.

In a particular embodiment of the invention, the corticosteroid is a clobetasol ester such as clobetasol 17-propionate, designated clobetasol propionate in the present application.

The amount of corticosteroid that can be incorporated according to the invention is from 0.0001% to 0.1% m/m, preferably from 0.001% to 0.05% m/m, and preferably from 0.0002% to 0.0005% m/m. This amount is preferably equal to 0.025% m/m.

According to a preferred embodiment of the invention, the composition is a film-forming solution, preferably a nail varnish, which also contains at least one promoter of absorption into the nail. Preferably, the composition contains two absorption promoters.

The expression “promoter of absorption into the nail” means pharmaceutically acceptable chemical compounds capable of increasing the permeability of the nail with respect to the active ingredients, to increase the kinetics of penetration of these active ingredients through the nail.

The absorption promoters according to the invention include urea, glycols, such as propylene glycol, butylene glycol, hexylene glycol, ethylene glycol or polyethylene glycols, glycol monoethers, such as the ethylene glycol monoethers marketed under the trademarks “Dowanol PM, DPM, TPM, PnB, PPH, DPnB, TPnB, PMA”, glycol polyethers such as ethoxydiglycol, propylene glycol dimethyl ether or dipropylene glycol dimethyl ether, dimethyl sulfoxide, amino acids and derivatives thereof such as N-acetyl-L-cysteine, and a mono- or polycarboxylated C₁ to C¹⁸, preferably C₁ to C₁₂, carboxylic acid and derivatives thereof such as hydroxymonocarboxylic acids, hydroxydicarboxylic acids, or the free acids, and also the lactones, the salts, the esters derived therefrom, caprolactam, dimethyl-acetamide and dimethylisosorbide. Other absorption promoters according to the invention are described in U.S. Pat. No. 6,455,592.

Among the C₁ to C₁₂ aliphatic carboxylic acids, and in particular hydroxyl acids, exemplary are methanoic acid, 2-methylbutanoic acid, propanoic acid, 2-methyl-propanoic acid, 2, 2-dimethylpropanoic acid, decanoic acid, octanoic acid, hex-2-enoic acid, heptanoic acid, 6-methylheptanoic acid, 3-ethylpentanoic acid, 3-chloropentanoic acid, 2-hydroxypropanoic acid, 2-chloro-4-hydroxyhexanoic acid, hexanedioic acid, octadecanoic acid, 4-oxopentanoic acid, 6-hydroxy-4-oxonanoic acid, 2-ketopropanoic acid, tartronic acid, malic acid, tartaric acid, glucaric acid, citric acid, lactic acid, glycolic acid, isocitric acid, tropic acid, 5-hydroxylauric acid and 3-hydroxy-4-methoxy-mandelic acid, or mixtures thereof.

In particular, the solutions according to the invention may comprise, as aliphatic carboxylic acid, lactic acid or citric acid, preferably lactic acid.

Preferably, the absorption promoters are the pairs urea/lactic acid or urea/N-acetyl-L-cysteine.

The urea is incorporated at a concentration of less than 15% by weight of urea relative to the weight of the nonvolatile part of the composition, in particular at a concentration of less than 14% by weight of urea relative to the weight of the non-volatile part of the composition, preferably from 7% to 14%, and more particularly from 9% to 13% by weight of urea relative to the weight of the non-volatile fraction of the composition; the lactic acid is incorporated in an amount of from 0.01% to 15% m/m, and preferably from 1% to 10%, in particular from 1% to 7%; and the N-acetyl-L-cysteine is incorporated in an amount of from 0.5% to 10% m/m, preferably from 1% to 7% m/m.

The weight proportion of ethoxydiglycol relative to the total weight of the composition is from 0.01% to 20% m/m, and preferably from 1% to 10%. As indicated above, the solutions according to the invention contain a solvent.

The solvents and cosolvents can be selected from the organic solvent family, and are class 3 solvents with a low toxic potential according to the ICH standards (Impurities: Guideline for Residual Solvents, International Conference of Harmonization), such as ethanol, isopropyl alcohol, acetone, methyl acetate, ethyl acetate, butyl acetate, alkyl methyl sulfoxides, such as dimethyl sulfoxide, 2-propanol, methyl isobutyl ketone, 1-butanol, dichloromethane or N-methyl-2-pyrrolidone, or mixtures thereof.

Among the solvents/cosolvents as described above, preferred are volatile organic solvents/cosolvents, and more preferably a mixture of ethanol and of at least one cosolvent selected from ethyl acetate and butyl acetate.

The solvents/cosolvents are included at the preferential concentrations ranging, respectively, from 10% to 90% to from 0% to 30% m/m, and more preferably ranging respectively from 10% to 80% to from 1% to 30% min.

Preferably, the vitamin D or derivatives thereof, preferably calcitriol, and the corticosteroid, preferably clobetasol propionate, are solubilized in the preferred solvent, i.e., ethanol.

Since the preparation of the compositions according to the invention requires the presence of at least one film-forming agent, the latter is preferably water-insoluble and selected from:
copolymers of monoalkyl esters of polyvinyl methyl ether and maleic acid, such as the butyl ester of polyvinyl methyl ether and maleic acid copolymer (butyl ester of PVM/MA copolymer) marketed under the trademark Gantrez ES 425 by ISP,
copolymers of acrylic and methacrylic acid esters with a low content of quaternary ammonium groups derived from acrylic acid, such as the acrylate and ammonium methacrylate copolymer (acrylate/ammonium methacrylate copolymer) marketed under the trademark Eudragit RL 100 by Rohm Pharma,
cellulose derivatives, such as the nitrocellulose or the ethylcellulose marketed by Aqualon,
polyurethane derivatives, such as the Avalures marketed by Noveon.

Also useful film-forming agents according to the present invention are polyvinylpyrrolidones and derivatives thereof, such as poly-I-vinyl-2-pyrrolidone, polyvinylpyrrolidone/vinyl acetate copolymer and vinyl-pyrrolidone/dimethylaminoethyl methacrylate copolymer, the copolymer derived from vinylpyrrolidone/acrylic acid and lauryl methacrylate, polysaccharides such as, in particular, chitosans and derivatives, gums such as guar gum, carrageenan gums, karaya gum or xanthan gum, polyvinyl alcohols, polyacrylamides, acrylic/methacrylic, polymethacrylate/butyl acrylate or acrylic/acrylate copolymers, cyanoacrylic polymers, or polyvinyl methyl ether/maleic anhydride copolymer.

The film-forming agents as described above are included at the preferential concentrations ranging from 0.01% to 50% m/m, preferably 5% to 30% m/m.

The compositions can also contain a plasticizer. The plasticizer is preferably included at concentrations ranging from 0.001% to 10.00% m/m, preferably from 0.5% to 5% m/m. Among such plasticizers, exemplary are phthalates, triacetates or citrates, or mixtures thereof. The plasticizer is preferably triacetin.

The film-forming solutions according to the invention can also comprise any additive normally used in the cosmetics or pharmaceutical field, such as sequestering agents, wetting agents, adhesion agents, spreading agents, antioxidants, sunscreens, preservatives, fillers, electrolytes, humectants, pigments, dyes, of usual bases or acids, which may be inorganic or organic, essential oils, cosmetic active agents, moisturizers, vitamins, essential fatty acids, or sphingolipids. Of course, one skilled in the art will take care to choose this or these optional additional compound (s) and/or the amount thereof, in such manner that the advantageous properties of the composition according to the invention are not, or are not substantially, impaired.

The present invention also features administration of the compositions as described above, as medicaments.

Finally, this invention features administration of a composition as defined above, in a therapeutic regime or regimen for the prevention or treatment of nail psoriasis.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

Composition:

| Starting Materials | Amounts as % m/m |
|---|---|
| Clobetasol propionate | 0.025 |
| Calcitriol | 0.0003 |
| Lactic acid | 4.00 |
| Urea | 2.50 |
| Gantrez ES-435 | 20.00 |
| Ethyl acetate | 17.00 |
| Butyl acetate | 6.00 |
| Ethanol | qs 100 |

### EXAMPLE 2

Composition:

| Starting Materials | Amounts as % m/m |
|---|---|
| Clobetasol propionate | 0.025 |
| Calcitriol | 0.0003 |
| Urea | 2.50 |
| N-acetyl-L-cysteine | 1.50 |
| Eudragit RL100 | 14.00 |
| Triacetin | 1.50 |
| Ethyl acetate | 17.00 |
| Butyl acetate | 6.00 |
| Ethanol | qs 100 |

### EXAMPLE 3

Composition:

| Starting Materials | Amounts as % m/m |
|---|---|
| Clobetasol propionate | 0.025 |
| Calcipotriol | 0.0003 |
| Lactic acid | 4.00 |
| Urea | 2.50 |
| Gantrez ES-435 | 20.00 |
| Ethyl acetate | 17.00 |
| Butyl acetate | 6.00 |
| Ethanol | qs 100 |

### EXAMPLE 4

Composition:

| Starting Materials | Amounts as % m/m |
|---|---|
| Clobetasol propionate | 0.025 |
| Calcitriol | 0.0003 |
| Urea | 2.50 |
| N-acetyl-L-cysteine | 1.50 |
| Poly-1-vinyl-2-pyrrolidone | 2.00 |
| Ethyl acetate | 17.00 |
| Butyl acetate | 6.00 |
| Ethanol | qs 100 |

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable film-forming solution useful for dermatological applications, comprising, as active agents solubilized therein, vitamin D or derivative thereof and a corticosteroid, formulated into a topically applicable, physiologically acceptable medium therefor.

2. The film-forming solution as defined by claim 1, comprising a nail varnish.

3. The film-forming solution as defined by claim 2, comprising a non-aqueous nail varnish.

4. The film-forming solution as defined by claim 1, said vitamin D or derivative thereof being selected from the group consisting of vitamin D₁, D₂, D₃ or D₄, calcipotriol, 25-hydroxyvitamin D₃, 1α-hydroxyvitamin D₃, calcitriol, 1α, 25, 26-trihydroxyvitamin D3, 1α, 23, 25-trihydroxyvitamin D3, 24, 25-dihydroxyvitamin D3, 1α, 25-dihydroxyvitamin D2, 1α-hydroxyvitamin D2, 1α, 24-dihydroxyvitamin D2 and 1α, 24-dihydroxyvitamin D3, and mixtures thereof.

5. The film-forming solution as defined by claim 4, comprising the vitamin D derivative calcitriol.

6. The film-forming solution as defined by claim 4, said corticosteroid being selected from the group consisting of clobetasone, clobetasone 17-butyrate, clobetasol, clobetasol 17-propionate, hydrocortisone, hydrocortisone 17-butyrate, cortisone, cortisone 21-acetate, prednisolone, prednisolone pivalate, miconazole, prednisone, triamcinolone, triamcinolone acetonide, methylprednisolone, fluometholone, fluocinolone, fluocinolone acetonide, desonide, betamethasone, betamethasone 21-acetate, betamethasone 17-adamantoate, betamethasone 17-benzoate, betamethasone 17-valerate, betamethasone 17,21-di-propionate and dexamethasone, and mixtures and derivatives thereof.

7. The film-forming solution as defined by claim 6, comprising the corticosteroid clobetasol propionate.

8. The film-forming solution as defined by claim 1, comprising at least one absorption promoter selected from the group consisting of urea, ethoxydiglycol, lactic acid and N-acetyl-L-cysteine.

9. The film-forming solution as defined by claim 8, comprising two absorption promoters selected from the pairs urea/lactic acid and urea/N-acetyl-L-cysteine.

10. The film-forming solution as defined by claim 1, comprising from 0.00001% to 0.1% of vitamin D or derivatives thereof by weight relative to the total weight of the solution.

11. The film-forming solution as defined by claim 10, comprising about 0.0003% of vitamin D or derivatives thereof by weight relative to the total weight of the solution.

12. The film-forming solution as defined by claim 10, comprising from 0.0001% to 0.1% of corticosteroid by weight relative to the total weight of the solution.

13. The film-forming solution as defined by claim 12, comprising about 0.025% of corticosteroid by weight relative to the total weight of the solution.

14. The film-forming solution as defined by claim 1, comprising at least one film-forming agent selected from the group consisting of poly-I-vinyl-2-pyrrolidone, butyl ester of polyvinyl methyl ether and maleic acid copolymer, and acrylate and ammonium methacrylate copolymer.

15. The film-forming solution as defined by claim 1, comprising ethanol and at least one cosolvent selected from ethyl acetate and butyl acetate.

16. The film-forming solution as defined by claim 15, wherein said vitamin D or derivative thereof is solubilized in ethanol.

17. The film-forming solution as defined by claim 15, wherein said corticosteroid is solubilized in ethanol.

18. A regime or regimen for the prevention or treatment of nail psoriasis, comprising topically applying onto the nail(s) of an individual in need of such treatment, a thus effective amount of the film-forming solution as defined by claim 1.
